(19) United States
(12) Patent Application Publication
(10) Pub. No.: US 2010/0055671 A1
(43) Pub. Date: Mar. 4, 2010

(54) Method for Determining Transduction Efficiency and Virus Dosage of Baculovirus

(75) Inventors: Yu-Chen Hu, Hsinchu (TW); Cnia-ni Yeh, Hsinchu (TW)

Correspondence Address: Cooper Legal Group LLC, 6505 Rockside Road, Suite 330, Independence, OH, 44131, US

(73) Assignee: National Tsing Hua University, Hsinchu (TW)

(21) Appl. No.: 12/203,436

(22) Filed: Sep. 3, 2008

(51) Int. Cl. C12Q 1/70

(52) U.S. Cl. 435/5

(57) ABSTRACT

A method for determining transduction efficiency of baculovirus includes: providing a recombinant baculovirus, in which the recombinant baculovirus includes an inducible promoter and a reporter gene positioned downstream the inducible promoter; adding the recombinant baculovirus to incubating environment of a mammalian cell for transduction; adding an inducer to promote expression of the reporter gene in the mammalian cell; and analyzing the percentage of the mammalian cell expressing the reporter gene to determine the transduction efficiency of the recombinant baculovirus. The method provides the ability to quantitatively analyze baculovirus transduction and is a simple and faster quantitative method applicable in transduction and other research study by including an inducible promoter and a reporter gene and thus prevents from imposing excessive metabolic burden to the cells. A method for determining virus dosage of baculovirus applied in genetic therapy is also disclosed.

### BACKGROUND OF THE INVENTION

1. Field of the Invention

The present invention relates to a method for determining baculovirus, and more particularly to a method for determining transduction efficiency and virus dosage of baculovirus.

2. Description of the Prior Art

In studies of virology and gene therapy, most researchers obtain the infectious titer (pfu/ml, in which pfu represents plaque forming units and is a measuring unit of virus infection) by end-point dilution analysis or plaque assay and then predict virus dosage based on the multiplicity of infection (MOI) of virus. MOI is defined as pfu per cell (pfu/cell), i.e. the ratio of infectious virus particles to the number of cells to be infected. For example, MOI=0.1 represents that it takes 10⁵ infectious virus particles to infect 10⁶ cells. The foregoing method takes a lot of time (7 to 10 days for end-point dilution analysis) and has too much bias due to different operators and time measuring with the result that it is difficult to obtain accurate infectious titer of virus.

On the other hand, Chan et al. (“Determination of the baculovirus transducing titer in mammalian cells.” Biotechnol. Bioeng. 93: 564-571, 2006) found that there is no significant difference in transducing titer and gene expression level even though the huge difference between the infectious titer of baculovirus, i.e. the infectious titer and MOI of baculovirus do not represent its transducing titer and can not be applied in determining the virus dosage for transduction. To sum up, there is yet no appropriate method available for determining the transducing titer of baculovirus to mammalian cells.

Chan et al (2006) have developed a method for determining transducing titer of baculovirus to solve the above-mentioned problem by adopting recombinant baculovirus Bac-CE for transduction. The recombinant baculovirus includes human cytomegalovirus immediate early (CMV-IE) promoter for modulating the expression level of green fluorescent protein (GFP), which functions as a reporter gene. The percentage of GFP+ cells is detected with a flow cytometer and the transducing titer of baculovirus is then determined. However, this system has been found with the following limitation. The CMV-IE promoter is a strong promoter in the mammalian cells and therefore keeps driving the over-expression of the reporter gene positioned downstream. In case of over-expressing another target protein, cell death may occur as a result of huge cellular resources taken. Therefore, the above-mentioned limitation limits the practical use of the invention of Chan et al.

Baculovirus has been adopted as a genetic vector in gene therapy for the past years. The virus dosage is a key experimental factor that controls the result of gene transduction. However, different batches of baculovirus have different transduction efficiency. A method that can be commonly practiced in mammalian cells and accurately determine virus dosage by analyzing the transduction efficiency of baculovirus is hence needed to enhance the reproducibility of experiments and prediction of experimental outcome.

In addition, there is no appropriate standard for screening gene vector applied in gene therapy. The transduction efficiency of baculovirus provides a screening standard that can be worked for evaluating an appropriate gene vector and is a current goal to work out.

To sum up, it is now a current goal to develop a method for determining the transduction efficiency and virus dosage of baculovirus.

### SUMMARY OF THE INVENTION

The present invention is directed to a method for determining transduction efficiency of baculovirus and to determine transducing titer of recombinant baculovirus in a simple and faster way by including an inducible promoter and a reporter gene and thus prevents from imposing excessive metabolic burden to the cells.

The present invention is also directed to a method for determining virus dosage of a recombinant baculovirus to determine the virus dosage of the recombinant baculovirus in a simple and faster way, to predict the transduction efficiency of the recombinant virus, and to obtain the same transduction efficiency in gene therapy.

According to an aspect, a method for determining transduction efficiency of a baculovirus includes providing a recombinant baculovirus, wherein the recombinant baculovirus includes an inducible promoter and a reporter gene positioned downstream the inducible promoter; adding the recombinant baculovirus to an incubating environment of a mammalian cell for transduction; adding an inducer to promote expression of the reporter gene in the mammalian cell; and analyzing the percentage of the mammalian cell expressing the reporter gene to determine the transduction efficiency of the recombinant baculovirus.

According to another aspect, a method for determining virus dosage of a baculovirus includes providing a recombinant baculovirus, wherein the recombinant baculovirus includes an inducible promoter and a reporter gene positioned downstream the inducible promoter; adding the recombinant baculovirus to an incubating environment of a mammalian cell for transduction; adding an inducer to promote expression of the reporter gene in the mammalian cell; analyzing the percentage of the mammalian cell expressing the reporter gene to determine the transduction efficiency of the recombinant baculovirus; and determining the virus dosage of the recombinant baculovirus based on the transduction efficiency of the recombinant baculovirus.

To sum up, the transducing titer of a baculovirus for mammalian cells transduction is evaluated by determining the transduction efficiency of the baculovirus and may be further applied in the prediction of virus dosage and expression level of target proteins in gene therapy. Thus, the baculovirus transduction may be quantitatively analyzed in a simple and faster way, and may be applied in transduction and other research thereof.

Other advantages of the present invention will become apparent from the following description taken in conjunction with the accompanying drawings wherein are set forth, by way of illustration and example, certain embodiments of the present invention.

### BRIEF DESCRIPTION OF THE DRAWINGS

The foregoing aspects and many of the accompanying advantages of this invention will become more readily appreciated as the same becomes better understood by reference to the following detailed description, when taken in conjunction with the accompanying drawings, wherein:

FIG. 1 is a flow chart illustrating an embodiment of the present invention;

FIG. 2 is a diagram illustrating a recombinant baculovirus according to an embodiment of the present invention;

FIG. 3 is a flow chart illustrating an embodiment of the present invention;

FIG. 4 is a diagram illustrating a recombinant baculovirus according to an embodiment of the present invention;

FIG. 5A to FIG. 5D are bar diagrams illustrating the experimental results according to an embodiment of the present invention;

FIG. 6 is a broken line graph illustrating the experimental results according to an embodiment of the present result; and

FIG. 7 is a bar diagram illustrating the experimental results according to an embodiment of the present invention.

### DESCRIPTION OF THE PREFERRED EMBODIMENT

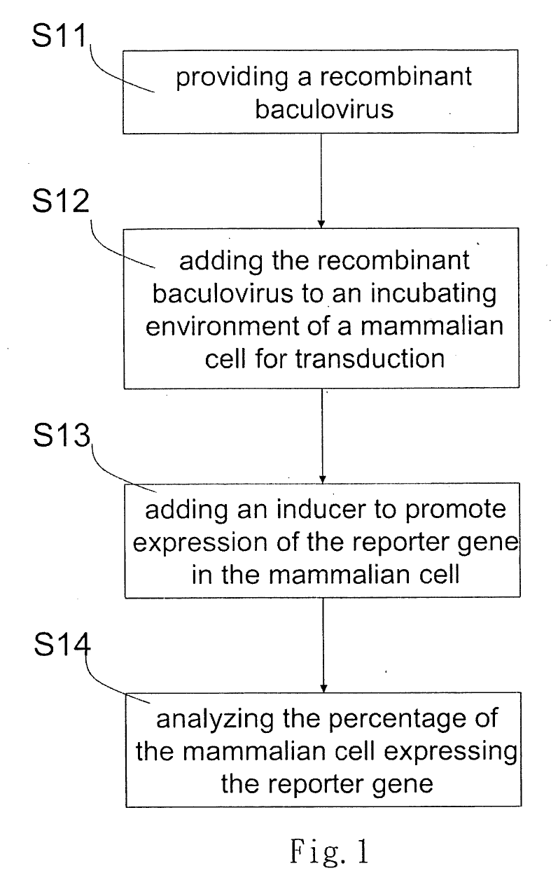

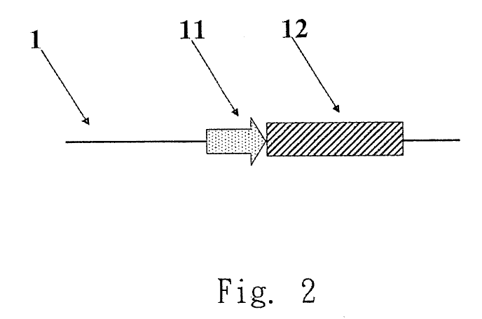

Referring to FIG. 1 and FIG. 2, FIG. 1 is a flow chart illustrating one preferred embodiment of the present invention, and FIG. 2 shows a recombinant baculovirus 1 according to an embodiment of the present invention.

Hereinafter, the method may be described as follows. At step S11, a recombinant virus 1 is provided. The recombinant virus includes an inducible promoter 11, and a reporter gene 12 positioned downstream the inducible promoter 11. In one embodiment, the inducible promoter 11 comprises a metallothionein promoter (hereinafter abbreviated as MT promoter), and the reporter gene 12 comprises an enhanced green fluorescence protein (hereinafter abbreviated as EGFP).

At step S12, the recombinant baculovirus 1 is added to the incubating environment of a mammalian cell for transduction.

At step S13, an inducer to promote expression of the reporter gene 12 is added into the mammalian cell. In one embodiment, the inducible promoter 11 comprises a MT promoter, and the corresponding inducer may be a zinc ion, a cadmium ion, a mercury ion, a copper ion, a bismuth ion, a nickel ion, cobalt ion, or any combination of the above-mentioned. In one embodiment, the inducer is a divalent zinc (Zn²⁺) ion because of its highest binding affinity with the MT promoter in the mammalian cell. In the above-mentioned embodiment, the Zn²⁺ ion (the inducer) binds to the MT promoter (the inducible promoter) to promote the expression of the downstream EGFP gene (the reporter gene).

It should be noted that an inducible promoter is adopted in the present invention which is modulated by an inducer and thus prevents the reporter gene from over-expression; therefore, less cellular resources are taken.

Next, the percentage of the mammalian cell expressing the reporter gene is analyzed to determine the transduction efficiency of the recombinant baculovirus (S14). In one embodiment, the percentage of the fluorescent mammalian cell is analyzed to determine the transduction efficiency of the recombinant baculovirus. The percentage of the fluorescent mammalian cells may be detected using a flow cytometer to determine the transduction efficiency of the recombinant baculovirus 1.

It should be noted that the above-mentioned embodiments are exemplary embodiments. For example, the inducible promoter 11 may be a GRE5 or a Gene switch system, and the corresponding inducer is a steroid; otherwise, the inducible promoter may be a Tet-on/Tet-off system, and the corresponding inducer is a tetracycline; furthermore, the inducible promoter 11 may be a dimerizer-regulated gene expression system, and the corresponding inducer is a rapamycin. In addition, the reporter gene 12 may be a luciferase.

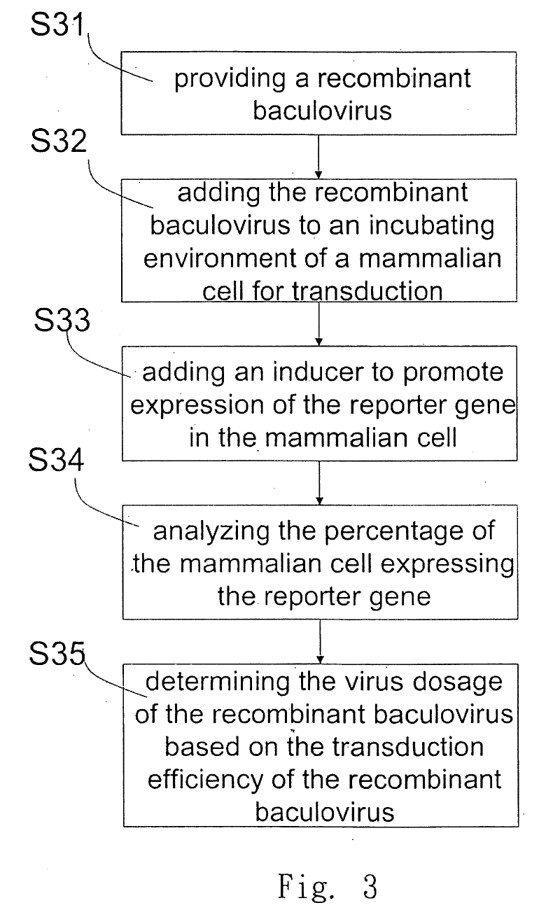

Referring to the FIG. 2 and FIG. 3, a method for determining virus dosage of a baculovirus is also illustrated as follows. First of all, a recombinant baculovirus 1 (S31) is provided, wherein the recombinant baculovirus 1 includes an inducible promoter 11 and a reporter gene 12 positioned downstream the inducible promoter 11. Next, the recombinant baculovirus 1 is added to the incubating environment of a mammalian cell for transduction (S32). Next, an inducer is added to promote expression of the reporter gene 12 in the mammalian cell (S33). Next, the percentage of the mammalian cell expressing the reporter gene 12 is analyzed to determine the transduction efficiency of the recombinant baculovirus 1 (S34). The steps S31 to S34 are the same as the above-mentioned steps S11 to S14 shown in the FIG. 1, and the detailed description is hence abbreviated.

Finally, the virus dosage is determined based on the transduction efficiency of the recombinant baculovirus 1 (S35). one embodiment of the present invention, the recombinant baculovirus is serially diluted to define a transducing titer (TT) of the recombinant baculovirus 1, in which the transducing titer is defined as the number of the transducible recombinant baculovirus per volume and calculated as:

$$T T = transduction efficiency ( \% ) \times cell number \times \frac{1}{0.1 ml}$$

The virus dosage may be defined as MOT (multiplicity of transduction), which is calculated as:

MOT=TT(TU/ml)×volume of virus(ml)/cell number.

The following descriptions of specific embodiments of the present invention have been presented for purposes of illustrations and description. They are not intended to be exclusive or to limit the invention to the precise forms disclosed, and obviously many modifications and variations are possible in light of the above teaching. It is intended that the scope of the invention be defined by the Claims appended hereto and their equivalents.

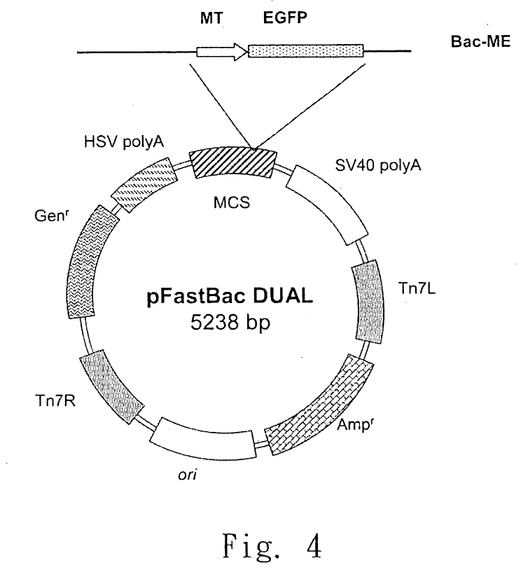

Referring to FIG. 4, in the present embodiment, a recombinant virus Bac-ME is constructed for the above-mentioned embodiments. Recombinant virus Bac-ME is constructed on the basis of GIBCO pFastBac-DUAL, in which strong promoters such as polyhedrin and p10 are removed for the subsequent construction. The Bac-ME includes a metallothionein promoter and an EGFP gene positioned downstream the metallothionein promoter; Tn7L and Tn7R function as the substrate sequence for the transposase which transposes the transgene cassette into the genome of DH10Bac E. coli cells. The metallothionein promoter (MT promoter) functions as an inducible promoter and originates from CHO cells (MT II promoter). The EGFP functions as a reporter gene and is positioned downstream the MT promoter. The EGFP expression in the mammalian cells is flexibly modulated by the MT promoter of the recombinant baculovirus, and is subsequently detected to determine the transduction efficiency of the recombinant baculovirus.

HeLa cells has been reported to be suitable for analyzing the transduction efficiency of the recombinant baculovirus and are hence adopted in the present invention, in which the cell density of HeLa cells is 2.5×10⁵ cells/ml. The transduction efficiency of the recombinant baculovirus is determined by detecting the percentage of GFP+ cells. In addition, Zn²⁺ ion has a better affinity with the MT promoter in mammalian cells, and ZnSO₄ is hence adopted as the inducer in the present invention. After 12 hours culturing of HeLa cells in a 6-well plate, the cells are transduced with 1:4 virus medium and incubating medium (e.g. 100 μl virus medium and 400 μl PBS) in the dark for 6 hours. The percentage GFP+ cells and fluorescent intensity of cells are then analyzed with a flow cytometer.

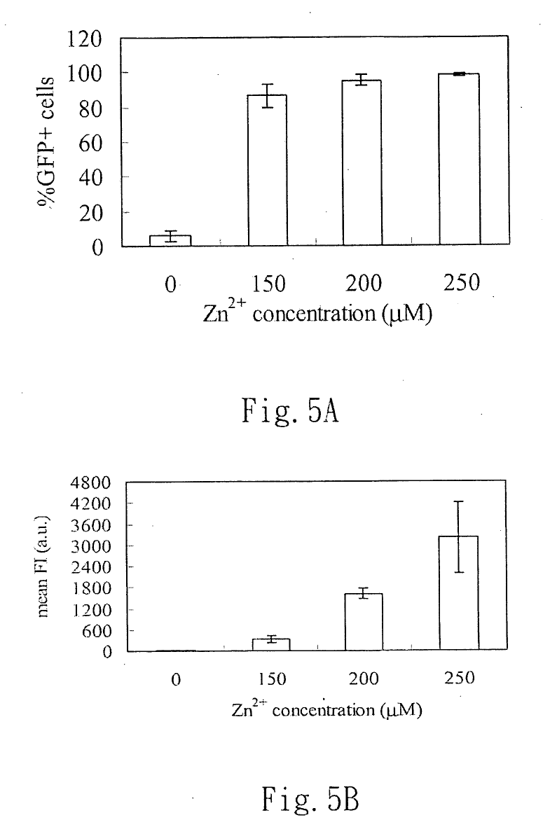

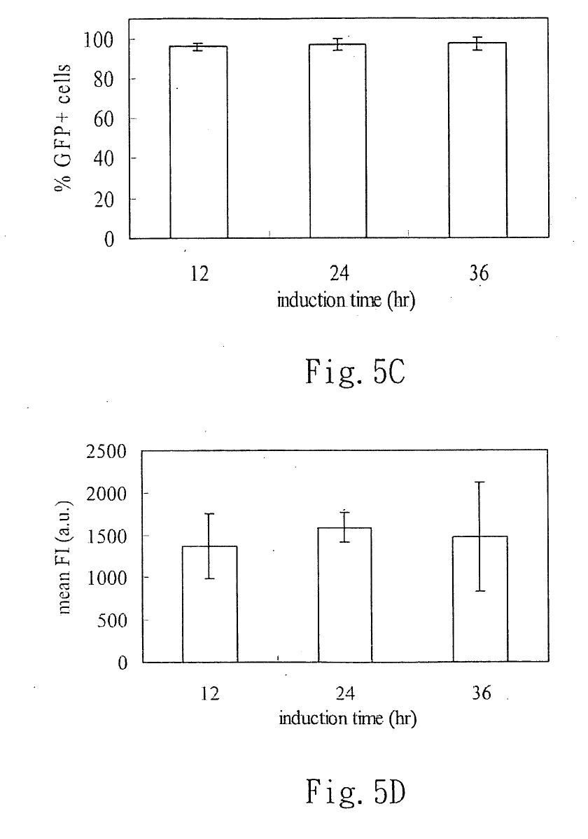

Referring to FIG. 5A, the percentage of GFP+ cells approximately reaches the highest at Zn²⁺ concentrations of 200 μM and 250 μM, in terms of highest transduction efficiency of the recombinant baculovirus to the mammalian cells. As shown in FIG. 5B, furthermore, the mean fluorescent intensity (FI) reaches the highest at a Zn²⁺ concentration of 250 μM.

In addition, it shows relatively low percentage of GFP+ cells (as illustrated in FIG. 5A) and mean fluorescent intensity (as illustrated in FIG. 5B) at the Zn²⁺ concentration of 0 μM, indicating a low background expression level of EGFP in the system.

Referring to FIGS. 5C and 5D, the Zn²⁺ concentration is chosen as 200 μM for appropriate resource planning of EGFP in the cells. The cells are transduced with 100 μl virus medium in the dark for 6 hours, and the percentage GFP+ cells and fluorescent intensity of cells are then analyzed. The percentage of GFP+ cells (in terms of transduction efficiency) and the fluorescent intensity reach a saturation point at an incubation time of 24 hours, as illustrated in FIGS. 5C and 5D.

The transducing titer is defined as a number of transducible recombinant baculovirus per unit volume, and is quantified and obtained from the transducing titer plot. The present method is validated by the following serial-dilution experiment, including: serial diluting different batches of virus medium (B1, B2, and B3) with incubating medium (TNM-FH with 10% FBS (fetal bovine serum)) in a volume factor of 2 (in the order of 2¹, 2², 2³ . . . , and 2¹¹); transducing HeLa cells with the diluted virus medium for 6 hours; incubating HeLa cells in the preferred induction condition (i.e. at the Zn²⁺ concentration of 200 μM and incubation time of 24 hours), and analyzing the percentage of GFP+ cells to determine the transduction efficiency of the different baculovirus. The transducing titer is obtained by diluting the recombinant baculovirus. As mentioned above, the transducing titer is defined as a number of transducible recombinant baculovirus per unit volume and obtained from:

TT=transduction efficiency(%)×cell number×1/0.1 ml

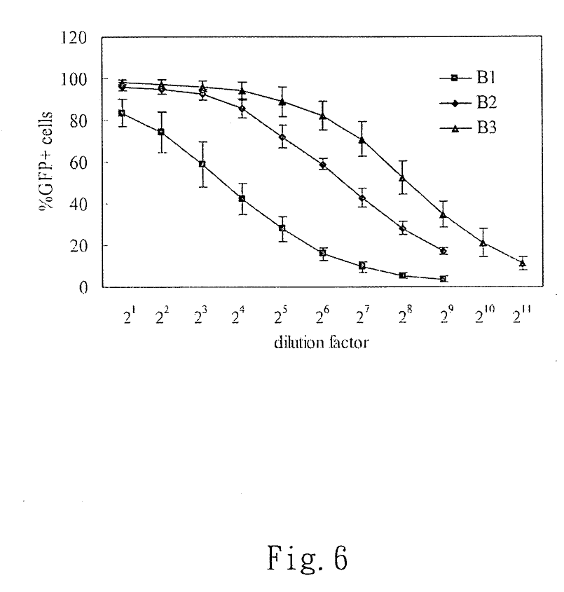

Referring to FIG. 6, it shows that the transducing titer plots of different batches of baculovirus do not overlap each other, and different batches of baculovirus are thus identified with the present method.

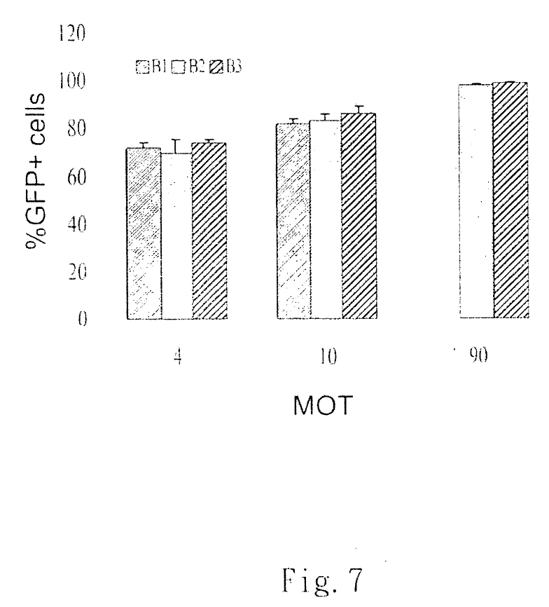

As mentioned above, the virus dosage is defined as the multiplicity of transduction (MOT) based on the transducing titer in the present invention. Therefore, the same MOT may be obtained from transducing titer of different batches of baculovirus, which is obtained from the transducing titer plots. The same transduction efficiency of different batches of baculovirus (B1, B2, and B3), as illustrated in FIG. 7, is obtained in the basis of the same MOT.

To sum up, the method for determining the transduction efficiency of baculovirus of the present invention does not adopt the conventional endpoint dilution method, and has advantages of being simple, fast, and accurate. In the application of gene therapy, the present invention can determine the virus dosage to predict the gene delivering efficiency therefore reproducible experiments are thus achieved.

While the invention is susceptible to various modifications and alternative forms, a specific example thereof has been shown in the drawings and is herein described in detail. It should be understood, however, that the invention is not to be limited to the particular form disclosed, but to the contrary, the invention is to cover all modifications, equivalents, and alternatives falling within the spirit and scope of the appended claims.

What is claimed is:

1. A method for determining transduction efficiency of a baculovirus, comprising:
providing a recombinant baculovirus, wherein the recombinant baculovirus comprises an inducible promoter and a reporter gene positioned downstream the inducible promoter;
adding the recombinant baculovirus into an incubating environment of a mammalian cell for transduction;
adding an inducer to promote expression of the reporter gene in the mammalian cell; and
analyzing a percentage of the mammalian cell expressing the reporter gene to determine a transduction efficiency of the recombinant baculovirus.

2. The method as claimed in claim 1, wherein the inducible promoter includes a metallothionein promoter (MT promoter).

3. The method as claimed in claim 2, wherein the inducer includes a zinc ion, a cadmium ion, a mercury ion, a copper ion, a bismuth ion, a nickel ion, a cobalt ion, or a combination thereof.

4. The method as claimed in claim 2, the inducer includes a divalent zinc ion.

5. The method as claimed in claim 1, the reporter gene includes an enhanced green fluorescence protein (EGFP) or a luciferase.

6. The method as claimed in claim 5, the transduction efficiency of the recombinant baculovirus is determined by analyzing a percentage of fluorescent mammalian cells.

7. The method as claimed in claim 1, wherein the inducible promoter is a GRE5 or a Gene switch system, and/or the inducer includes a steroid.

8. The method as claimed in claim 1, wherein the inducible promoter includes a Tet-on/Tet-off system, and/or the inducer includes tetracycline.

9. The method as claimed in claim 1, wherein the inducible promoter is a dimerizer-regulated gene expression system, and/or the inducer is a rapamycin.

10. A method for determining virus dosage of a baculovirus, comprising:
providing a recombinant baculovirus, wherein the recombinant baculovirus comprises an inducible promoter and a reporter gene positioned downstream the inducible promoter;
adding the recombinant baculovirus into an incubating environment of a mammalian cell for transduction;
adding an inducer to promote expression of the reporter gene in the mammalian cell;
analyzing a percentage of the mammalian cell expressing the reporter gene to determine a transduction efficiency of the recombinant baculovirus; and
determining a virus dosage of the recombinant baculovirus based on the transduction efficiency of the recombinant baculovirus.

11. The method as claimed in claim 10, wherein the inducible promoter includes a metallothionein promoter (MT promoter).

12. The method as claimed in claim 11, wherein the inducer includes a zinc ion, a cadmium ion, a mercury ion, a copper ion, a bismuth ion, a nickel ion, a cobalt ion, or a combination thereof.

13. The method as claimed in claim 11, the inducer includes a divalent zinc ion.

14. The method as claimed in claim 10, the reporter gene includes an enhanced green fluorescence protein (EGFP) or a luciferase.

15. The method as claimed in claim 14, the transduction efficiency of the recombinant baculovirus is determined by analyzing a percentage of fluorescent mammalian cells.

16. The method as claimed in claim 10 further comprising:
serially diluting the recombinant baculovirus to define a transducing titer (TT) of the recombinant baculovirus, wherein the transducing titer is defined as the number of the transducible recombinant baculovirus per volume and calculated by:
$$T T = transduction efficiency ( \% ) \times cell number \times \frac{1}{0.1 ml} ; and$$
defining the virus dosage as MOT (multiplicity of transduction), which is calculated by:
MOT=TT(TU/ml)×volume of virus(ml)/cell number

17. The method as claimed in claim 10, wherein the inducible promoter includes a GRE5 (glucocorticoid response elements 5) or a Gene switch system, and/or the inducer includes a steroid.

18. The method as claimed in claim 10, wherein the inducible promoter is a Tet-on/Tet-off system, and/or the inducer includes tetracycline.

19. The method as claimed in claim 10, wherein the inducible promoter is a dimerizer-regulated gene expression system, and/or the inducer includes rapamycin.
